(19) United States
(12) Patent Application Publication
(10) Pub. No.: US 2009/0082679 A1
(43) Pub. Date: Mar. 26, 2009

(54) CARDIAC MONITORING SYSTEM

(75) Inventor: Scott Matthew Chetham, Del Mar, CA (US)

Correspondence Address: SHAY GLENN LLP, 2755 CAMPUS DRIVE, SUITE 210, SAN MATEO, CA, 94403, US

(73) Assignee: Impedance Cardiology Systems, Inc., Menlo Park, CA (US)

(21) Appl. No.: 11/629,804

(22) PCT Filed: Jun. 21, 2005
(86) PCT No.: PCT/AU2005/000893

§ 371 Date: Dec. 15, 1916

(30) Foreign Application Priority Data
Jun. 21, 2004 (AU) 200403334
Oct. 26, 2004 (AU) 200406181

(51) Int. Cl. A61B 5/053; A61B 5/0295

(52) U.S. Cl. 600/508; 600/547

(57) ABSTRACT

A method of analyzing cardiac functions in a subject using a processing system. The method includes causing one or more electrical signals to be applied to the subject using a first set of electrodes, the one or more electrical signals having a plurality of frequencies. The method includes determining an indication of electrical signals measured across a second set of electrodes applied to the subject in response to the applied one or more signals. Following this, and for a number of sequential time instances, the method includes determining from the indicating data and the one or more applied signals, an instantaneous impedance value at each of the plurality of frequencies, and determining, using the using instantaneous impedance values, an intracellular impedance parameter. The intracellular impedance parameter over at least one cardiac cycle is the used to determine one or more parameters relating to cardiac function.

### BACKGROUND OF THE INVENTION

The present invention relates to a method and apparatus for monitoring biological parameters, and in particular to a method and apparatus for measuring cardiac function in a subject using bioelectric impedance.

### DESCRIPTION OF THE PRIOR ART

The reference to any prior art in this specification is not, and should not be taken as, an acknowledgment or any form of suggestion that the prior art forms part of the common general knowledge.

It is estimated that coronary heart disease will become the single biggest public health problem in the world by 2020. The treatment of coronary heart disease and other cardiovascular diseases therefore represents and increasingly large health and economic burden throughout the world in the coming years.

Cardiac output (CO), which can be defined as the amount of blood ejected by the ventricles of the heart per minute (measured in litres per minute), is governed by the metabolic demands of the body, and therefore reflect the status of the entire circulatory system. For this reason measurement of cardiac output is an essential aspect of haemodynamic monitoring of patients with heart disease or who are recovering from various forms of cardiovascular disease or other medical treatments.

One existing technique for determining cardiac function which has been developed is known as impedance cardiography (IC). Impedance cardiography involves measuring the electrical impedance of a subject's body using a series of electrodes placed on the skin surface. Changes in electrical impedance at the body's surface are used to determine changes in tissue volume that are associated with the cardiac cycle, and accordingly, measurements of cardiac output and other cardiac function.

A complication in impedance cardiography is that the baseline impedance of the thorax varies considerably between individuals, the quoted range for an adult is 20Ω-48Ω at a frequency between 50 kHz-100 kHz. The changes in impedance due to the cardiac cycle are a relatively small (0.5%) fraction of the baseline impedance, which leads to a very fragile signal with a low signal to noise ratio.

Accordingly, complex signal processing is required to ensure measurements can be interpreted.

An example of this is described in International patent publication no W02004/032738. In this example, the responsiveness of a patient to an applied current is modeled using the equivalent circuit shown in FIG. 1. The equivalent circuit assumes that:
direct current is conducted through the extracellular fluid only since the reactance of the cell membrane will be infinite;
an applied alternating current is conducted through the extracellular and intracellular pathways in a ratio dependent on the frequency of the applied signal.

Accordingly, the equivalent circuit includes an intracellular branch formed from a capacitance C representing the capacitance of the cell membranes in the intracellular pathway and the resistance R₁ representing the resistance of the intracellular fluid. The circuit also includes an extracellular branch formed from resistance RE which represents the conductive pathway through the tissue.

W02004/032738 operates based on the assumption that the cardiac cycle will only have an impact on the volume of extracellular fluid in the patient's thorax, and therefore that cardiac function can be derived by considering changes in the extracellular component of the impedance. This is achieved by applying an alternating current at a number of different frequencies. The impedance is measured at each of these frequencies and then extrapolated to determine the impedance at zero applied frequency, which therefore corresponds to the resistance R_{E}. This is then determined to be solely due to the extracellular fluid component and hence can be used to determine attributes of cardiac function, such as stroke volume.

However, in practice the impedance at zero frequency would not be due solely to extracellular fluids but would be influenced by a number of other factors. In particular, cells do not act as a perfect capacitor and accordingly, the intracellular fluid will contribute to the impedance at a zero applied frequency.

A further issue in W02004/032738 is that the process determines the impedance at zero applied frequency using the “Cole model”. However, again this assumes idealized behavior of the system, and consequently does not accurately model a subject's bioimpedance response. Consequently cardiac parameters determined using these techniques tend to be of only limited accuracy.

### SUMMARY OF THE INVENTION

In a first broad form the present invention provides a method of analyzing cardiac functions in a subject, the method including, in a processing system:
a) causing one or more electrical signals to be applied to the subject using a first set of electrodes, the one or more electrical signals having a plurality of frequencies;
b) determining an indication of electrical signals measured across a second set of electrodes applied to the subject in response to the applied one or more signals;
c) for a number of sequential time instances:

i) determining from the indicating data and the one or more applied signals, an instantaneous impedance value at each of the plurality of frequencies;

ii) determining, using the instantaneous impedance values, an intracellular impedance parameter; and,
d) determining, using the intracellular impedance parameter over at least one cardiac cycle, one or more parameters relating to cardiac function.

Typically the impedance parameter is a variable intracellular resistance parameter.

Typically the method includes, in the processing system:
a) determining, using the instantaneous impedance values, at least one impedance value; and,
b) determining the intracellular impedance parameter using the at least one impedance value and a predetermined equation.

Typically the predetermined equation is:

$${R}_{1} = \frac{{R}_{var}}{{( {τ}_{Y} {ω}_{Ym} )}^{- α}}$$

Typically the at least one impedance value includes at least one of:
a) the impedance at zero frequency;
b) the impedance at infinite frequency; and,
c) the impedance at a characteristic frequency.

Typically method includes, in the processing system, determining the intracellular impedance parameter is determined using a CPE model.

Typically the method includes, in the processing system, and for impedances determined at a time instance:
a) fitting a function to the instantaneous impedance values; and,
b) using the fitted function to determine the intracellular impedance parameter.

Typically the method includes, in the processing system:
a) fitting a function to the instantaneous impedance values;
b) determining any outlier instantaneous impedance values;
c) for any outlier instantaneous impedance values:

i) removing the instantaneous impedance value;

ii) recalculating the function; and,

iii) using the recalculated function if the recalculated function is a better fit for the instantaneous impedance values.

Typically the method includes, in the processing system, using the fitted function to determine one or more impedance values.

Typically the function includes at least one of:
a) a polynomial fitted using a curve fitting algorithm; and,
b) a function based on a Wessel plot.

Typically the method includes, in the processing system:
a) determining an indication of one or more subject parameters; and,
b) using the one or more subject parameters to determine the one or more parameters relating to cardiac function.

Typically the method includes, in the processing system, determining one or more parameters relating to cardiac function using the equation:

$$CO = {k}_{1} {{c}_{1} ( \frac{\vert {( \frac{d {R}_{var} ( t )}{d t} )}_{MAX} \vert}{{Z}_{0}} )}^{n} * {( \frac{1}{{T}_{RR}} )}^{m} \times {T}_{LVE}$$

Where:

i) CO denotes cardiac output (litres/min),

ii) k₁ is an optional population specific correction factor based on one or more subject parameters, such as at least the height and weight, but can also include distance between the electrodes and age;

iii) c₁ is an optional calibration coefficient used to convert the units from Ohmic units to litres (which may be uniquely defined at manufacture for each monitoring device used to implement the method),

iv) Z₀ is an optional baseline Impedance measured at the characteristic frequency (between 10 Ohms and 150 Ohms),

v) TRR is the interval between two R waves obtained from the ECG (found from the ECG or impedance or conductance data),

vi) TLVE is left ventricular ejection time (measured from either the conductance or impedance curve or preferably a combination of other physiological measurement techniques) and

vii) n(range −4>n<4) and m(range −4>m<4) are optional constants.

Typically the method includes, processing electrical signals measured across a second set of electrodes applied to the subject to perform at least one of:
a) removal of respiratory effects;
b) extraction of ECG signals; and,
c) removing unwanted signals.

Typically the method includes, in the processing system, displaying an indication of at least one of:
a) impedance values;
b) one or more intracellular impedance parameter values; and,
c) one or more parameters relating to cardiac function.

Typically the method includes, in the processing system, determining at least one of:
a) stroke volume;
b) cardiac output;
c) cardiac index;
d) stroke index;
e) systemic vascular resistance/index;
f) acceleration;
g) an acceleration index;
h) velocity;
i) velocity index;
j) thoracic fluid content;
k) left ventricular ejection time;
l) pre-ejection period;
m) systolic time ratio;
n) left cardiac work/index;
o) heart rate; and,
p) mean arterial pressure.

Typically the intracellular impedance parameter models at least resistance changes caused by the re-orientation of cellular components of the subject's blood over the cardiac cycle.
In a second broad form the present invention provides apparatus for analyzing cardiac functions in a subject, the apparatus including a processing system for:
a) causing one or more electrical signals to be applied to the subject using a first set of electrodes, the one or more electrical signals having a plurality of frequencies;
b) determining an indication of electrical signals measured across a second set of electrodes applied to the subject in response to the applied one or more signals;
c) for a number of sequential time instances:

i) determining from the indicating data and the one or more applied signals, an instantaneous impedance value at each of the plurality of frequencies;

ii) determining, using the instantaneous impedance values, an intracellular impedance parameter; and,
d) determining, using the intracellular impedance parameter over at least one cardiac cycle, one or more parameters relating to cardiac function.

Typically the impedance parameter is a variable intracellular resistance parameter.

Typically the apparatus includes:

a) a signal generator coupled to the processing system for generating electrical signals to be applied to the subject; and,

b) a sensor for sensing electrical signals across the subject.

Typically the signal generator is a current generator.

Typically the sensor is a voltage sensor.

Typically the apparatus includes a number of electrodes for coupling the signal generator and the sensor to the subject.

Typically the processing system is coupled to at least one of the signal generator and the sensor via a wireless connection.

Typically the sensor includes an analogue to digital converter.

Typically the processing system performs the method of the first broad form of the invention.

### BRIEF DESCRIPTION OF THE DRAWINGS

An example of the present invention will now be described with reference to the accompanying drawings, in which:

FIG. 1 is a schematic of an example of an equivalent circuit used to model the conduction characteristics of biological tissue;

FIG. 2 is a flowchart of an example of a process for determining cardiac function;

FIGS. 3A and 3B are schematics of an example of the effects of blood flow on blood cell orientation;

FIG. 4 is a schematic of a second example of an equivalent circuit used to model the conduction characteristics of biological tissue;

FIG. 5 is a schematic of an example of apparatus for determining cardiac function;

FIGS. 6A to 6C are a flowchart of a second example of a process for determining cardiac function;

FIG. 7 is an example of a graph of impedance plotted against frequency for an impedance measurement;

FIG. 8 is an example of a Wessel diagram of susceptance plotted against conductance; and

FIG. 9 is an example of three plots depicting the time varying impedance of the thorax, the level of impedance change due to cardiac function and an ECG.

### DETAILED DESCRIPTION OF THE PREFERRED EMBODIMENTS

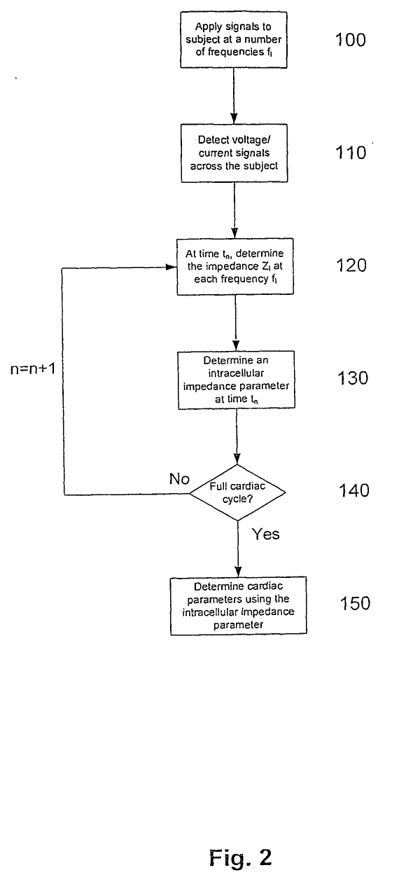

An example of a process for determining parameters of cardiac function relating to a subject is described with reference to FIG. 2.

In particular at step 100, alternating electrical signals are applied to the subject at a number of different frequencies fᵢ, with electrical signals across the subject being detected at each of the respective fᵢ, at step 110. The nature of the signals applied and detected will depend on the implementation as will be described below.

At step 120, at a first time instance tₙ the impedance Zᵢ at each frequency fᵢ is determined. At step 130, the impedance is used to determine an intracellular impedance parameter at the time tₙ. In one example, this is achieved utilizing an appropriate model, such as a CPE (constant phase element) model, which will be described in more detail below.

This is performed for a number of sequential time instance tₙ, tₙ₊₁, tₙ₊₂ until it is determined that a complete cardiac cycle has been analyzed at step 140. This may be achieved by monitoring appropriate ECG signals, or alternatively simply by processing sufficient time instances to ensure that a cardiac cycle has been detected.

At step 150, the intracellular impedance parameter, and in one example, changes in the intracellular impedance parameter, is used to determine cardiac parameters.

This technique takes into account that the impedance fluctuation of the thorax during the cardiac cycle is dependent on both changes in blood volume and changes in the impedance in the blood itself.

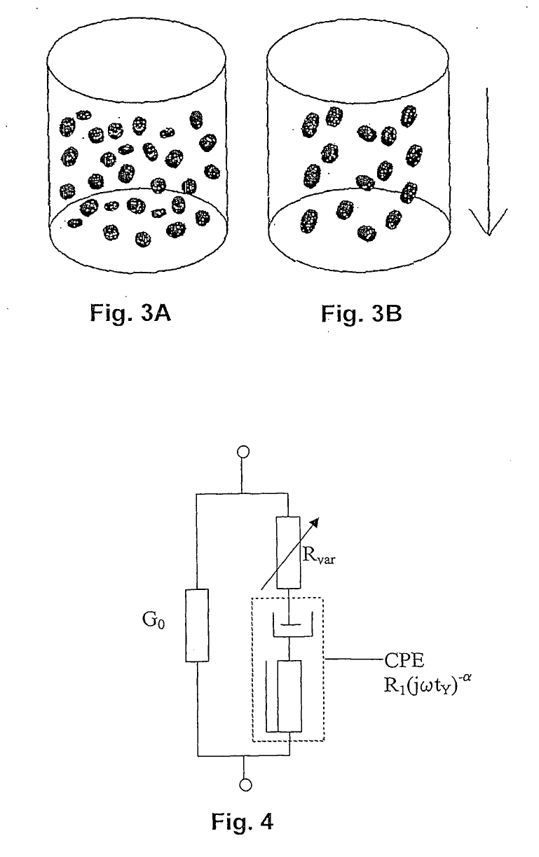

Blood is a suspension of erythrocytes, with a high resistivity, and other cells in a conducting fluid called plasma. The erythrocytes of stationary blood are randomly orientated as shown in FIG. 3A, and hence the resistivity of stationary blood is isotropic. Due to their biconcave shape erythrocytes tend to align themselves in flowing blood with their axes parallel to the direction of flow as shown in FIG. 3B. Accordingly, the resistivity of flowing blood is anisotropic.

The anisotropy of the resistivity is due to the longer effective path length for the current travelling normal to the axis of the vessel compared with the current flowing parallel to the vessel. As a result, the resistance of the intracellular fluid alters depending on the orientation of the erythrocytes, and hence depends on the flow of blood.

Furthermore, the extent of the anisotropy is shear-rate dependent since the orientation of the erythrocytes is influenced by the viscous forces in flowing blood. As a result, the resistivity is in turn also dependent on the flow rate.

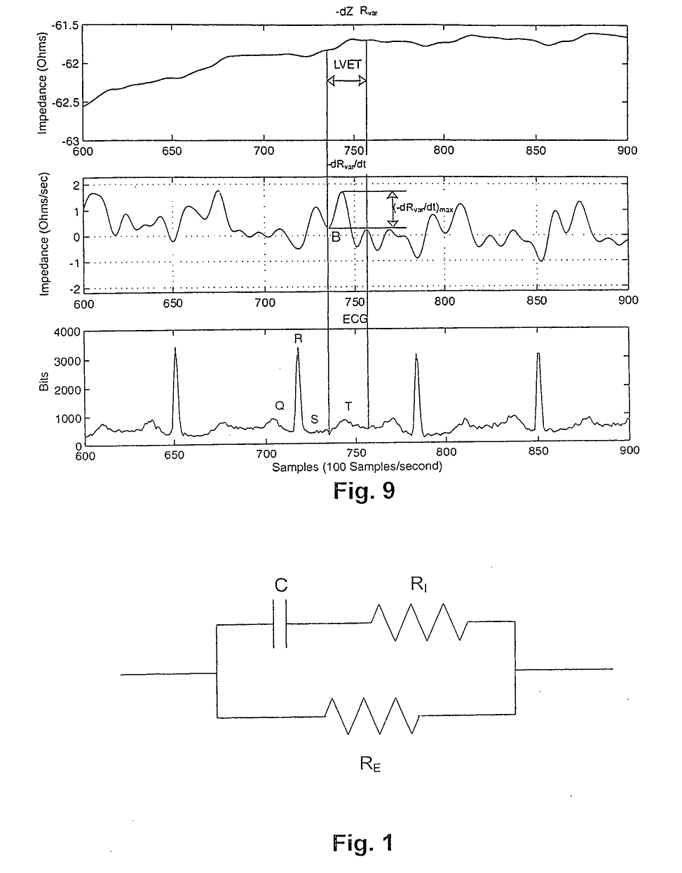

It is therefore possible to take this into account by determining cardiac function on the basis of intracellular parameters, as opposed to using extracellular impedance parameters as in the prior art. This can therefore be achieved using the equivalent circuit shown in FIG. 1, and by using the impedance measurements to determine the impedance parameters based on the capacitance C and the resistance R₁ of the intracellular branch.

Thus, in this instance, the impedance measurements can be used to determine values for the intracellular resistance R₁ and the capacitance C, for example, by determining values of R₀ and R_{∞}, and then using these to solve the Cole equation using appropriate mathematical techniques.

In this instance however, modeling the resistivity as a constant value does not accurately reflect the impedance response of a subject, and in particular does not accurately model the change in orientation of the erythrocytes, or other relaxation effects.

To more successfully model the electrical conductivity of blood, an improved CPE based model can be used as will now be described with respect to FIG. 4.

In this example, to accurately determine the characteristic impedance, and interpret the contribution of cardiac effects to the impedance, an equivalent circuit based on a free conductance parallel model is used, as shown in FIG. 4. Such a model can also be created in a series form and the parallel model is shown here for illustration.

In this example, the circuit includes an extracellular conductance G₀ that represents the conductance of electrical current through the extracellular fluid. The intracellular conduction path includes a constant phase element (CPE) represented as the series connection of a frequency dependent conductance, and a frequency dependent capacitance.

The two equations below define a general CPE:

$$\begin{matrix}{Y}_{CPE} = {( ωτ )}^{m} ( {G}_{ωτ = 1} + j {B}_{ωτ = 1} ) & ( 1 ) \\ {ϕ}_{cpe} = \frac{arctan B}{G} & ( 2 )\end{matrix}$$

where:

Y_{CPE} is the admittance of the CPE and

φ_{cpe} is the phase of the CPE.

In this equation τ represents a frequency scale factor and, ωτ is dimensionless.

The parameter m defines the extent of the frequency dependence of the admittance of the CPE Y_{CPE} and the frequency scale factor with τ. It is known that for biological tissue m is in the range of 0≦m≦1.

In one example, the CPE is in accordance with Fricke's law (CPE_{F}) although other forms of CPE could be used. It is usual practice to use the exponent symbol α(m=α) for Fricke CPE's.

In order to make the model compatible with relaxation theory, the series ideal resistor is changed to a free resistor parameter Rᵥₐᵣ a so that the characteristic time constant τ_{z} will be a dependent parameter.

The result is that the conductance of the circuit can be expressed as follows:

$$Y = {G}_{0} + \frac{1}{{R}_{var} + {{R}_{1} ( {jωτ}_{z} )}^{- a}} \qquad ( 3 )$$

$${τ}_{Ym} = \frac{1}{{ω}_{Ym}} = {{τ}_{Y} ( \frac{{R}_{1}}{{R}_{var}} )}^{\frac{1}{- a}} \qquad ( 4 )$$

Here τ_{Ym} is a new characteristic time constant. The subscript m is used to identify the new variable from the previous variables and is consistent with the nomenclature know to those skilled in the art.

By putting a nominal fixed value to the time constant τ_{y} it is possible to follow the CPE by calculating the R₁ using the equation.

$${R}_{1} = \frac{{R}_{var}}{{( {τ}_{Y} {ω}_{Ym} )}^{- a}} \qquad ( 5 )$$

In this instance, the variable resistance parameter Rᵥₐᵣ is dependent on the orientation of the erythrocytes and as a result, changes in Rᵥₐᵣ can be used to determine the rate of flow of blood within the a subject. Consequently, it is possible to determine information regarding cardiac output, or the like.

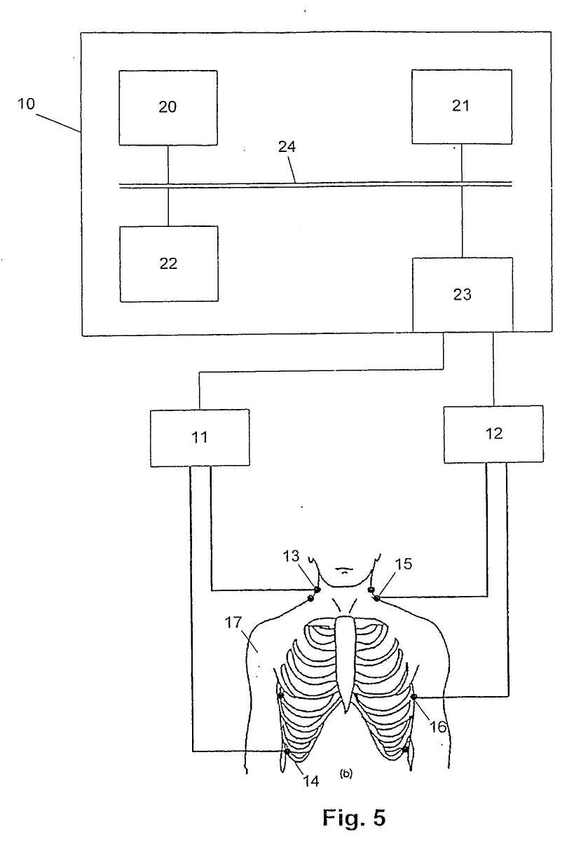

An example of apparatus suitable for performing an analysis of a subject's bioelectric impedance to determine cardiac function will now be described with reference to FIG. 5.

As shown the apparatus includes a processing system 10 having a processor 20, a memory 21, an input/output (I/O) device 22 and an interface 23 coupled together via a bus 24. The processing system is coupled to a signal generator 11 and a sensor 12 as shown. In use the signal generator 11 and the sensor 12 are coupled to respective electrodes 13, 14, 15, 16, as shown.

In use, the processing system 10 is adapted to generate control signals, which causes the signal generator 11 to generate an alternating signal which is applied to a subject 17, via the electrodes 13, 14. The sensor 12 then determines the voltage or current across the subject 17 and transfers appropriate signals to the processing system 10.

Accordingly, it will be appreciated that the processing system 10 may be any form of processing system which is suitable for generating appropriate control signals and interpreting voltage data to thereby determine the subject's bioelectrical impedance, and optionally determine the cardiac parameters.

The processing system 10 may therefore be a suitably programmed computer system, such as a laptop, desktop, PDA, smart phone or the like. Alternatively the processing system 10 may be formed from specialized hardware. Similarly, the I/O device may be of any suitable form such as a touch screen, a keypad and display, or the like.

It will be appreciated that the processing system 10, the signal generator 11 and the sensor 12 may be integrated into a common housing and therefore form an integrated device. Alternatively, the processing system 10 may be connected to the signal generator 11 and the sensor 12 via wired or wireless connections. This allows the processing system 10 to be provided remotely to the signal generator 11 and the sensor 12. Thus, the signal generator 11 and the sensor 12 may be provided in a unit near, or worn by the subject 17, whilst the processing system is situated remotely to the subject 17.

In practice, the outer pair of electrodes 13, 14 are placed on the thoracic and neck region of the subject and an alternating signal is applied at a plurality of frequencies either simultaneously or in sequence, (two are sufficient but at least three are preferred with five or more being particularly advantageous) in the range 2-2000 kHz. However the applied waveform may contain more frequency components outside of this range.

In the preferred implementation the applied signal is a frequency rich voltage from a voltage source clamped so it does not exceed the maximum allowable patient auxiliary current. The signal can either be constant current, impulse function or a constant voltage signal where the current is measured so it does not exceed the maximum allowable patient auxiliary current.

A potential difference and/or current are measured between an inner pair of electrodes 16, 17. The acquired signal and the measured signal will be the superposition of signals at each of the applied frequencies and the potentials generated by the human body, such as the ECG.

Optionally the distance between the inner pair of electrodes may be measured and recorded. Similarly, other parameters relating to the subject may be recorded, such as the height, weight, age, sex, health status, and other information, such as current medication, may also be recorded.

The acquired signal is demodulated to obtain the impedance of the system at the applied frequencies. One suitable method for demodulation is to use a Fast Fourier Transform (FFT) algorithm to transform the time domain data to the frequency domain. Another technique not requiring windowing of the measured signal is a sliding window FFT. Other suitable digital and analog demodulation techniques will be known to persons skilled in the field.

Impedance or admittance measurements are determined from the signals at each frequency by comparing the recorded voltage and current signal. The demodulation algorithm will produce an amplitude and phase signal at each frequency.

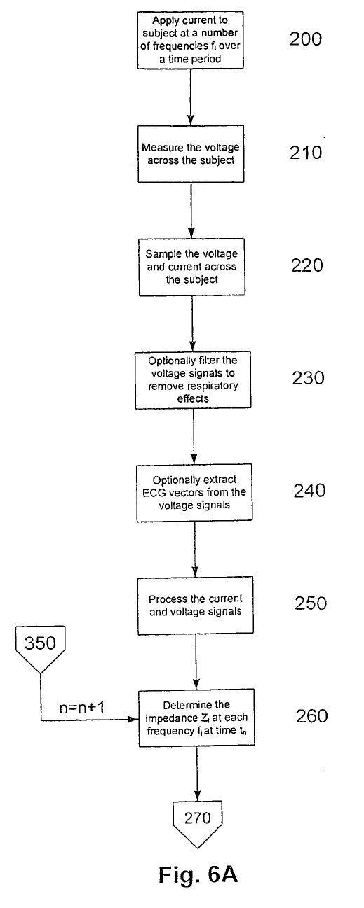

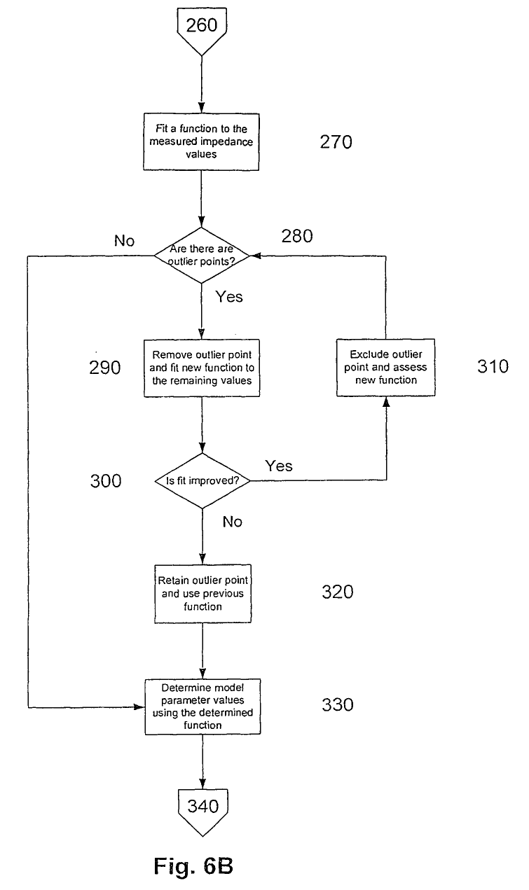

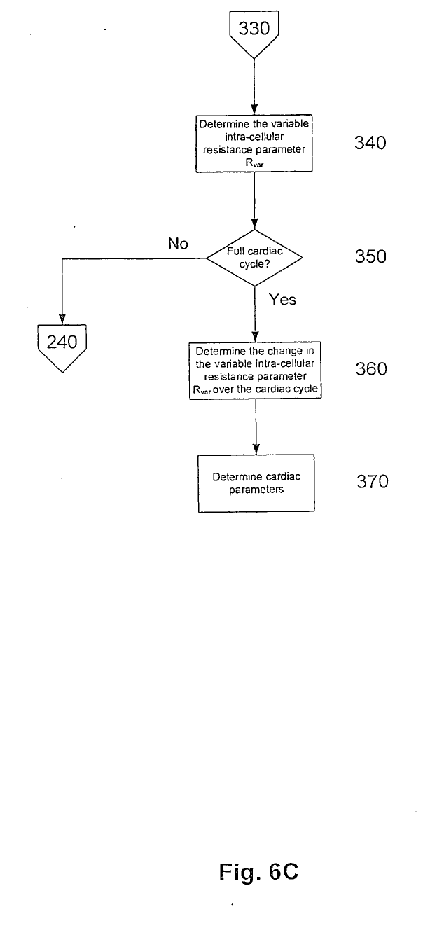

An example of the process of measuring a subject's bioelectric impedance and then interpreting this will be described in more detail with reference to FIGS. 6A to 6C.

At step 200 the processing system 10 generates predetermined control signals causing the signal generator 11 to apply current signals to the subject 17 at a number of frequencies fᵢ, over a time period T. The current signals applied to the subject 17 may be provided at the frequencies fᵢ sequentially, or simultaneously, by superposing a number of signals at each corresponding frequency fᵢ.

It will be appreciated that the control signals are typically generated in accordance with data stored in the memory 21 and this can allow a number of different current sequences to be used, with selection being made via the I/O device 22, or via another appropriate mechanism.

At step 210 the sensor 12 measures the voltage across the subject 17. In this regard, the voltage signals will typically be analogue signals and the sensor 12 will operate to digitise these, using an analogue to digital converter (not shown).

At step 220 the processing system 10 samples the signals from the signal generator 11 and the sensor 12, to thereby determine the current and voltage across the subject 17.

At step 230, a filter is optionally applied to the voltage signals at step 230 to remove respiratory effects, which typically have a very low frequency component in line with the patient's rate of breathing. It will be appreciated that filtering may be achieved by the sensor 12 or the processing system 10, depending on the implementation.

At step 240 ECG vectors are optionally extracted from the voltage signals. This can be achieved as the ECG signals typically have a frequency in the region 0 Hz to 100 Hz, whereas the impedance signals are in the region of 5 kHz to 1 MHz. Accordingly, the ECG signals may be extracted by any suitable technique, such as demodulation, filtering or the like.

At step 250 the signals may also undergo additional processing. This can be performed, for example, by further filtering the signals to ensure that only signals at the applied frequencies fᵢ, are used in impedance determination. This helps reduce the effects of noise, as well as reducing the amount of processing required.

At step 260, the current and voltage signals sampled at time tₙ to determine the impedance Zᵢ at each frequency fᵢ.

At step 270 a function is fitted to the impedance values.

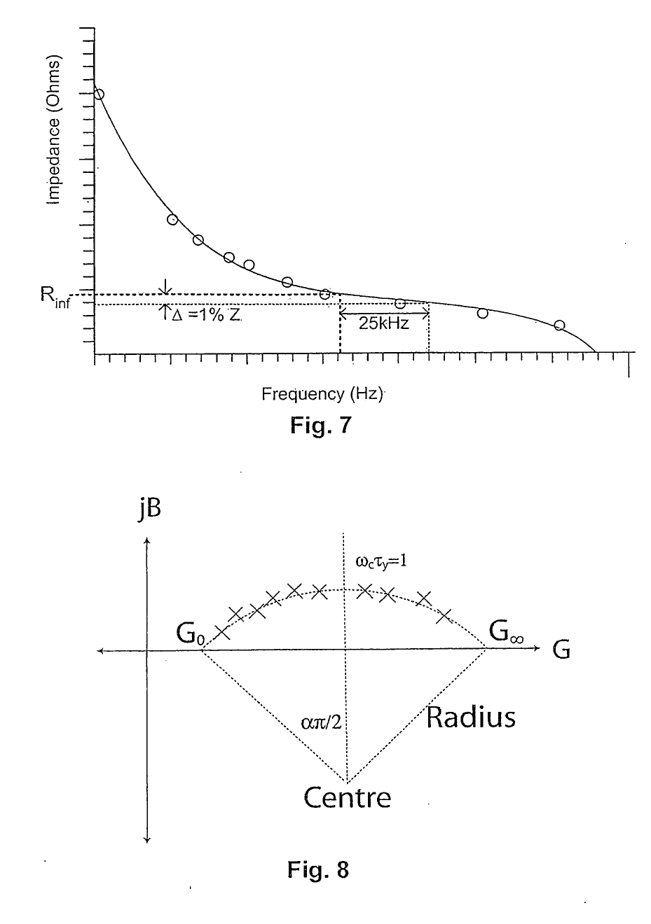

An example of this is shown in FIG. 7, which shows an example of the appearance of the impedance data and function when plotted against frequency. It will be appreciated that the plot is for the purpose of example only, and in practice the processing system 10 will not necessarily generate a plot. In the case of the frequency verses the impedance plot shown in FIG. 7, the function is typically a polynomial and in particular in this example is a sixth order polynomial.

Alternatively a Wessel plot may be used as shown in FIG. 8, as will be described in more detail below.

In practice noise elimination may be necessary to accurately fit a function to the data. In one example, elimination of noise at certain frequencies can be performed by initially fitting a function to the measured data and then systematically removing outlier points from the data set and re-fitting the function to the reduced data set.

Accordingly, at step 280 the processing system 10 operates to determine if there are outlier points, which are considered to be points that are greater than a predetermined distance from the determined function.

It will be appreciated that the function used, and the determination of outlier points may be achieved utilizing standard mathematical techniques.

If it is determined that there are outlier points, these are removed from the data set and a new function fitted to the remaining values at step 290. At step 290 the processing system 10 determines if the fit is improved and if so the outlier point is excluded from the data set permanently with the new function being assessed at step 310. This is repeated until all outliers that affect the data are removed.

If it is determined that the fit is not improved at step 300 the outlier is retained and the previous function used at step 320.

If there are no outliers, or once outliers have been excluded from the data set, the plot is then used to determine values from Rₒ and R_{∞} using the determined function.

In one example, the function is used to calculate R₀ and R_{∞}. Alternatively, this can be used to determine the impedance at the characteristic frequency.

For example, in the case of the function shown in FIG. 7, R_{∞} can be determined by finding the impedance at the start of the pseudo-plateau, i.e. a relatively flat portion, on the curve of FIG. 7. In the illustrative embodiment the pseudo plateau is identified using a rule-based approach.

In this approach the function is analyzed to find the frequency where impedance (Z) changes (ΔZ) by less than 1% with a frequency increase of 25 kHz. The resistance or impedance Z measured at this frequency is identified as R_{∞}and represents resistance of the circuit if an infinitely high frequency was applied. Other methods of determining this pseudo-plateau region may be known to those skilled in the art.

Similarly, the impedance at zero applied frequency R₀ can be determined as the value at which the function would intercept the y-axis.

If a “Wessel” plot type function is used, as shown in FIG. 8, this approach uses an arc, which allows the characteristic impedance to be determined. In this example, the apex of the arc in the complex Wessel plane no longer corresponds to the nominal value of τ_{y}, but to τ_{Ym} as given by the above equation.

Additionally a can be determined from the angle subtended by the arcuate locus from R₀ to R_{∞}. By comparing this to m determined from susceptance data, this allows whether the Fricke criteria for relaxation phenomena of biological materials is met. In the event that they are equal or within a predetermined range of each other, then the Wessel diagram method may be applied with reasonable accuracy. In the event that m and a are not sufficiently close in value then the function fitting approach described above is a more appropriate method for determining the quantities of interest for the free conductance model.

At step 340 the processing system 10 uses the values of either R₀ to R_{∞}, or the characteristic impedance, together with equation (5) to determine the intracellular impedance parameter, which in this example is the intracellular variable resistance parameter Rᵥₐᵣ

As an alternative to determining values of R₀, R_{∞}, or the characteristic impedance Z_{c}, the equation (5) can alternatively be solved mathematically, for example by using a number of different impedance values at different frequencies fi to solve a number of simultaneous equations. These values can be based on directly measured values, although preferably these are values determined from the fitted function, to thereby take into account the impedance response across the range of applied frequencies fᵢ.

At step 350 it is determined if a full cardiac cycle has been completed and if not the process returns to step 240 to analyze the next time instance tₙ₊₁.

At step 360, once a full cardiac cycle has been completed, the processing system 10 operates to determine the change in the intracellular resistance parameter Rᵥₐᵣ over the cardiac cycle before using this to determine cardiac parameters at step 370.

A typical plot of the time varying impedance obtained by the present method is shown in FIG. 9.

In FIG. 9 the raw impedance data is plotted against time (measured by sample number) in the top graph. This graph includes the impedance from all time varying impedance components in the thoracic cavity including variation in blood volume, blood cell orientation and changes due to respiration.

The centre graph of FIG. 9 depicts the rate of change of impedance attributable to cardiac function of a patient. The graph was generated by removing the low frequency components from the top graph and obtaining the rate of change of impedance from the remaining data.

As will be appreciated by those skilled in the art additional measurements can also be incorporated into the present method or conducted simultaneously. For example, the inner electrodes can also be used to record ECG vectors. In order to generate more ECG vectors more inner electrode combinations are required. The outer electrodes can also be used to record the ECG vectors. The processing unit, or the operator, can automatically or manually select the most appropriate ECG vector. An external ECG monitor can also be connected or alternatively a separate module can be incorporated into the invention with additional electrodes to calculate the ECG vectors.

The ECG can advantageously be used to aid in the determination of cardiac events. An example ECG output is depicted in the lower graph of FIG. 9.

To calculate certain cardiac parameters from the impedance waveform, fiducial points must also be suitably identified. The ECG data and/or other suitable physiological measurement techniques may be employed to aid this process.

Other physiological parameters that could be used to assist in identifying fiducial points in the cardiac cycle include invasive/non-invasive blood pressure, pulse oximetry, peripheral bioimpedance measurements, ultrasound techniques and infrared/radio frequency spectroscopy. Such techniques can be used singularly or in a plurality to optimally determine cardiac event timing.

In one example an artificial neural network or weighted averages to determine the cardiac events as identified by conductance measurements combined with other methods of physiological measures offer an improved method of identifying these points. In the present example the start and end of left ventricular ejection are indicated by the vertical lines on the graphs of FIG. 9. The time between these points is the left ventricle ejection time (LVET).

These fiducial points can be used to obtain impedance values of interest. For example the maximum rate of change in the intracellular resistance value Rᵥₐᵣ over left ventricle ejection which is indicated on the central graph of FIG. 9 as:

$${( \frac{d {R}_{var} ( t )}{d t} )}_{MAX}$$

Measures of cardiac function can then be determined from this data. For example, the following method can be used to calculate blood velocity and stroke volume. The present example uses impedance measures to calculate cardiac output. However the same functions can be described using admittance or a combination of the two. The following formula can be used to calculate cardiac output:

$$CO = {k}_{1} {{c}_{1} ( \frac{\vert {( \frac{d {R}_{var} ( t )}{d t} )}_{MAX} \vert}{{Z}_{0}} )}^{n} * {( \frac{1}{{T}_{RR}} )}^{m} \times {T}_{LVE}$$

Where:
CO denotes cardiac output (litres/min),

$${( \frac{d {R}_{var} ( t )}{d t} )}_{max}$$

is as indicated on FIG. 9;
k₁ is an optional population specific correction factor based on one or more subject parameters, such as at least the height and weight, but can also include distance between the electrodes and age;
c₁ is an optional calibration coefficient used to convert the units from Ohmic units to litres (which may be uniquely defined at manufacture for each monitoring device used to implement the method),
Z₀ is an optional baseline Impedance measured at the characteristic frequency (between 10 Ohms and 150 Ohms),
T_{RR} is the interval between two R waves obtained from the ECG (found from the ECG or impedance or conductance data),
T_{LVE} is left ventricular ejection time (measured from either the conductance or impedance curve or preferably a combination of other physiological measurement techniques) and
n(range −4>n<4) and m (range −4>m<4) are optional constants.

The person skilled in the art will be able to determine appropriate values for these constants dependent upon the patient and situation in which the method is applied.

Whilst the example described above has been described in the context of providing determining cardiac output of the heart, embodiments of the present invention can be applied to determine other measures of cardiac performance, including but not limited to, stroke volume, cardiac index, stroke index, systemic vascular resistance/index, acceleration, acceleration index, velocity, velocity index, thoracic fluid content, left ventricular ejection time, Pre-ejection period, systolic time ratio, left cardiac work/index, heart rate and mean arterial pressure.

Persons skilled in the art will appreciate that numerous variations and modifications will become apparent. All such variations and modifications which become apparent to persons skilled in the art, should be considered to fall within the spirit and scope that the invention broadly appearing before described.

1. A method of analyzing cardiac functions in a subject, the method including, in a processing system:
a) causing one or more electrical signals to be applied to the subject using a first set of electrodes, the one or more electrical signals having a plurality of frequencies;
b) determining an indication of electrical signals measured across a second set of electrodes applied to the subject in response to the applied one or more signals;
c) for a number of sequential time instances:
i) determining from the indicating data and the one or more applied signals, an instantaneous impedance value at each of the plurality of frequencies;
ii) determining, using the instantaneous impedance values, an intracellular impedance parameter; and,
d) determining, using the intracellular impedance parameter over at least one cardiac cycle, one or more parameters relating to cardiac function.

2. A method according to claim 1, wherein the impedance parameter is a variable intracellular resistance parameter.

3. A method according to claim 1, wherein the method includes, in the processing system:
e) determining, using the instantaneous impedance values, at least one impedance value; and,
f) determining the intracellular impedance parameter using the at least one impedance value and a predetermined equation.

4. A method according to claim 3, wherein the predetermined equation is:
$${R}_{1} = \frac{{R}_{var}}{{( {τ}_{Y} {ω}_{Ym} )}^{- a}}$$

5. A method according to claim 3, wherein the at least one impedance value includes at least one of:
g) the impedance at zero frequency;
h) the impedance at infinite frequency; and,
i) the impedance at a characteristic frequency.

6. A method according to claim 1, wherein method includes, in the processing system, determining the intracellular impedance parameter is determined using a CPE model.

7. A method according to claim 1, wherein the method includes, in the processing system, and for impedances determined at a time instance:
j) fitting a function to the instantaneous impedance values; and,
k) using the fitted function to determine the intracellular impedance parameter.

8. A method according to claim 7, wherein the method includes, in the processing system:
l) fitting a function to the instantaneous impedance values;
m) determining any outlier instantaneous impedance values;
n) for any outlier instantaneous impedance values:
i) removing the instantaneous impedance value;
ii) recalculating the function; and,
iii) using the recalculated function if the recalculated function is a better fit for the instantaneous impedance values.

9. A method according to claim 7, wherein the method includes, in the processing system, using the fitted function to determine one or more impedance values.

10. A method according to claim 7, wherein the function includes at least one of:
o) a polynomial fitted using a curve fitting algorithm; and,
p) a function based on a Wessel plot.

11. A method according to claim 1, wherein the method includes, in the processing system:
q) determining an indication of one or more subject parameters; and,
r) using the one or more subject parameters to determine the one or more parameters relating to cardiac function.

12. A method according to claim 1, wherein the method includes, in the processing system, determining one or more parameters relating to cardiac function using the equation:
$$CO = {k}_{1} {{c}_{1} ( \frac{\vert {( \frac{d {R}_{var} ( t )}{d t} )}_{MAX} \vert}{{Z}_{0}} )}^{n} * {( \frac{1}{{T}_{RR}} )}^{m} \times {T}_{LVE}$$
where:
i) CO denotes cardiac output (litres/min),
ii) k₁ is an optional population specific correction factor based on one or more subject parameters, such as at least the height and weight, but can also include distance between the electrodes and age;
iii) c₁ is an optional calibration coefficient used to convert the units from Ohmic units to litres (which may be uniquely defined at manufacture for each monitoring device used to implement the method),
iv) Z₀ is an optional baseline Impedance measured at the characteristic frequency (between 10 Ohms and 150 Ohms),
v) TRR is the interval between two R waves obtained from the ECG (found from the ECG or impedance or conductance data),
vi) TLVE is left ventricular ejection time (measured from either the conductance or impedance curve or preferably a combination of other physiological measurement techniques) and
vii) n(range −4>n<4) and m(range −4>m<4) are optional constants.

13. A method according to claim 1, wherein the method includes, processing electrical signals measured across a second set of electrodes applied to the subject to perform at least one of:
b) removal of respiratory effects;
c) extraction of ECG signals; and,
d) removing unwanted signals.

14. A method according to claim 1, wherein the method includes, in the processing system, displaying an indication of at least one of:
e) impedance values;
f) one or more intracellular impedance parameter values; and,
g) one or more parameters relating to cardiac function.

15. A method according to claim 1, wherein the method includes, in the processing system, determining at least one of:
h) stroke volume;
i) cardiac output;
j) cardiac index;
k) stroke index;
l) systemic vascular resistance/index;
m) acceleration;
n) an acceleration index;
o) velocity;
p) velocity index;
q) thoracic fluid content;
r) left ventricular ejection time;
s) pre-ejection period;
t) systolic time ratio;
u) left cardiac work/index;
v) heart rate; and,
w) mean arterial pressure.

16. A method according to claim 1, wherein the intracellular impedance parameter models at least resistance changes caused by the re-orientation of cellular components of the subject's blood over the cardiac cycle.

17. Apparatus for analyzing cardiac functions in a subject, the apparatus including a processing system for:
x) causing one or more electrical signals to be applied to the subject using a first set of electrodes, the one or more electrical signals having a plurality of frequencies;
y) determining an indication of electrical signals measured across a second set of electrodes applied to the subject in response to the applied one or more signals;
z) for a number of sequential time instances:
i) determining from the indicating data and the one or more applied signals, an instantaneous impedance value at each of the plurality of frequencies;
ii) determining, using the instantaneous impedance values, an intracellular impedance parameter; and,
aa) determining, using the intracellular impedance parameter over at least one cardiac cycle, one or more parameters relating to cardiac function.

18. Apparatus according to claim 17, wherein the impedance parameter is a variable intracellular resistance parameter.

19. Apparatus according to claim 17, the apparatus including:
bb) a signal generator coupled to the processing system for generating electrical signals to be applied to the subject; and,
cc) a sensor for sensing electrical signals across the subject.

20. Apparatus according to claim 19, wherein the signal generator is a current generator.

21. Apparatus according to claim 19, wherein the sensor is a voltage sensor.

22. Apparatus according to claim 19, wherein the apparatus includes a number of electrodes for coupling the signal generator and the sensor to the subject.

23. Apparatus according to claim 19, wherein the processing system is coupled to at least one of the signal generator and the sensor via a wireless connection.

24. Apparatus according to claim 19, wherein the sensor includes an analogue to digital converter.

25. (canceled)
